SEQUENCE LISTING (4 sequences)

SEQ ID NO: 1 - DNA, 19 bp, Artificial Sequence
  Synthetic
cgtgtgtttgagaacattg

SEQ ID NO: 2 - DNA, 19 bp, Artificial Sequence
  Synthetic
ctgatgtgtgagaagcgga

SEQ ID NO: 3 - protein, 8 aa, Gallus gallus
SIINFEKL

SEQ ID NO: 4 - DNA, 21 bp, Artificial Sequence
  Synthetic
atggcgtggaatctgcaaggg

(19) United States
(12) Patent Application Publication
(10) Pub. No.: US 2016/0361391 A1
(43) Pub. Date: Dec. 15, 2016

(54) REDUCTION OF TRAF1 LEVELS COMBINED WITH NUTRIENT STRESS FOR TREATING LYMPHOCYTE-RELATED CANCERS

(71) Applicants: THE GOVERNING COUNCIL OF THE UNIVERSITY OF TORONTO, Toronto (CA); UNIVERSITY HEALTH NETWORK, Toronto (CA)

(72) Inventors: Ali Abdul Sater, Toronto (CA); Ann Josephine McPherson, Toronto (CA); Tania Helen Watts, Toronto (CA); Mark Minden, Toronto (CA); Rob Laister, Toronto (CA)

(21) Appl. No.: 15/121,563

(22) PCT Filed: Mar. 6, 2015
(86) PCT No.: PCT/CA2015/000144

§ 371 Date: Aug. 25, 2016

Related U.S. Application Data
(60) Provisional application No. 61/949,033, filed on Mar. 6, 2014.

(51) Int. Cl. A61K 38/46; C12N 15/113; G01N 33/574; A61K 31/7088

(52) U.S. Cl. CPC A61K 38/465; A61K 31/7088; C12N 15/1137; G01N 33/57426; C12Y 305/01001; C12N 2310/122; C12N 2320/31; G01N 2333/525

(57) ABSTRACT

The present disclosure provides methods and uses for treating lymphocyte related cancers comprising administering a nutrient stress-inducing agent in combination with an agent that lowers the levels of or inhibits TRAF1. Also provided are methods of determining treatment for a subject with a lymphocyte related cancer and assays for identifying a PKN1 inhibitor.

### RELATED APPLICATIONS

This application claims the benefit of priority to U.S. provisional application No. 61/949,033 filed Mar. 6, 2014, the content of which is incorporated herein by reference in its entirety.

### FIELD

The present disclosure relates to methods and uses for treating lymphocyte-related cancers. In particular, the present disclosure relates to the reduction of TRAF1 directly or indirectly through PKN1 in combination with a nutrient stress-inducing agent.

### BACKGROUND

### TNFR Family Members and TNFR Associated Factors (TRAFs) and Cellular Survival

Tumour necrosis factor receptor (TNFR) family members play critical roles in controlling cellular life and death.¹ Members of the TNFR family that lack death domains, including 4-1BB and CD40, signal cellular survival by recruiting TNFR associated factors (TRAFs).² TRAF1 is a signalling adaptor that lacks signalling function on its own, but forms a 1:2 heterotrimer with another TRAF protein, TRAF2, and this complex then recruits downstream signalling molecules to induce activation of the NF-κB signalling pathway (FIG. 1).³ Previous work has shown that TRAF1 is important for survival of T lymphocytes during an immune response⁴ and that TRAF1 plays a critical role in signalling downstream of the TNFR family member 4-1BB.^{5, 6} TRAF1 is critical to sustain TRAF2-dependent signalling downstream of the TNFR family member CD40 in B cells and dendritic cells^{7, 8} CD40 contributes to the activation, proliferation and survival of mature B cells and their neoplastic counterparts.⁹

TNFRs, TRAF1 and Cancer.

TRAF1 is overexpressed in approximately half of all B cell lineage related cancers with highest expression in the most refractory B-cell chronic lymphocytic leukemia.¹⁰ Moreover, single nucleotide polymorphisms in TRAF1 are linked to non-Hodgkin's lymphoma.¹¹ Several TRAF1-recruiting TNFR family members can be expressed on lymphomas, including CD30, CD40 and the EBV protein LMP1. Many human malignancies including B-cell chronic lymphocytic leukemia, Burkitt's lymphoma cells and B cell lineage non-Hodgkin's lymphomas, exhibit constitutive CD40 signalling thus allowing them to autonomously maintain continuous proliferative activity.^{12, 13, 14, 15} LMP1 normally signals constitutively in a ligand independent manner¹⁶ and CD30 when overexpressed also shows ligand independent signalling.¹⁷ TRAF1 expression is also required for lymphomagenesis in a spontaneous mouse tumour model involving constitutively active NF-κB2.¹⁸ Thus targeting TRAF1 degradation in human lymphoma cells has the potential to improve disease outcome. However, to date, a direct link between TRAF1 levels and prognosis in human cancer has not been made.

Nutrient Stress-Induced by Erwinase for Treatment of Leukemia and Lymphoma.

Lymphomas, much like other tumours, have high energy and metabolic demands. Proliferating lymphocytes and lymphomas increase their anabolic metabolism driven by glycolysis as the major source of ATP.^{19, 20} In addition to the requirement for high glucose uptake, proliferating cancer cells require high levels of glutamine, which contributes to energy production as well as biosynthesis and redox control.^{21, 22, 23, 24} Erwinase is a licensed drug that is currently used for treatment of certain leukemias and lymphomas.²⁵ The anti-tumour activity of Erwinase resides in its enzyme (asparaginase) activity, where it not only depletes the circulating pool of L-asparagine but also that of glutamine, resulting in inhibition of protein synthesis and creating nutrient stress in the tumour microenvironment.²⁶

Regulation of TRAF2 Degradation by Nutrient Stress when TRAF1 is Limiting.

TRAF2 is critical for NF-κB activation downstream of most TNFRs, including CD40, CD30, and LMP1 on B lymphomas. Published studies have shown that during signaling through the TNFR family members 4-1 BB, CD40, or TNFR2, the absence of TRAF1 can lead to decreased levels of TRAF2, leading to the idea that TRAF1 is required to stabilize TRAF2^{7, 8, 27, 28}.

### SUMMARY

The present disclosure provides a method of treating a lymphocyte related cancer comprising administering a nutrient stress-inducing agent in combination with an agent that lowers the levels of or inhibits TRAF1. Also provided herein is a use of a nutrient stress-inducing agent in combination with an agent that lowers the levels of or inhibits TRAF1 for treating a lymphocyte related cancer. Further provided is a use of a nutrient stress-inducing agent in combination with an agent that lowers the levels of or inhibits TRAF1 in the preparation of a medicament for treating a lymphocyte related cancer. Even further provided is a nutrient stress-inducing agent in combination with an agent that lowers the levels of or inhibits TRAF1 for use in treating a lymphocyte related cancer.

In an embodiment, the lymphocyte related cancer is of B-cell origin. In another embodiment, the lymphocyte related cancer is of T-cell origin. In another embodiment, the lymphocyte related cancer is lymphoma, such as Burkitt's lymphoma, B-cell lineage non-Hodgkins Lymphoma or Diffuse Large B cell Lymphoma (DCBL), or leukemia, such as chronic lymphocytic leukemia (CLL).

In an embodiment, the subject has been identified as having a TRAF1 high expressing lymphocyte related cancer.

In another embodiment, the nutrient stress-inducing agent is asparaginase or arginase. In a particular embodiment, the asparaginase is Erwinase.

The agent that lowers the levels of or inhibits TRAF1 can inhibit TRAF1 expression or activity directly or indirectly. In one embodiment, the agent that lowers the levels of or inhibits TRAF1 is an antisense or shRNA to TRAF1. In a particular embodiment, the agent that lowers the levels of or inhibits TRAF1 is an shRNA that comprises the nucleotide sequence as shown in SEQ ID NO:1 or a variant thereof.

In another embodiment, the agent that lowers the levels of or inhibits TRAF1 is a PKN1 inhibitor. In one embodiment, the PKN1 inhibitor is an antisense or shRNA to PKN1. In a particular embodiment, the shRNA comprises the nucleotide sequence as shown in SEQ ID NO:2 or a variant thereof.

In another aspect, there is a method of determining treatment for a lymphocyte-related cancer in a subject comprising:

a) determining whether the lymphocyte-related cancer is TRAF1 low or TRAF1 high by comparing to a control;

b) treating the subject with a nutrient stress-inducing agent if the subject is TRAF1 low; and

c) treating the subject with a nutrient stress-inducing agent in combination with an agent that lowers the levels of or inhibits TRAF1 if the subject is TRAF1 high.

In another embodiment, the present disclosure provides a method of determining treatment for a lymphocyte related cancer in a subject comprising determining whether the lymphocyte related cancer is TRAF1 low or TRAF1 high; wherein if the cancer is TRAF1 low it is indicative of treating the cancer with a nutrient stress-inducing agent and if the cancer is TRAF1 high it is indicative of treating the cancer with a nutrient stress-inducing agent in combination with an agent that lowers the levels of or inhibits TRAF1.

In an embodiment, the control is a TRAF1 high control from the same type of tumour or from a pool of positive tumours of the same type.

In another embodiment, the TRAF1 high control is a reference standard, optionally obtained from a panel of banked samples of the same class of tumour and optionally determined relative to Raji or Daudi cell lines. In an embodiment, the standard is historical data for a pool of patients and optionally this data is continually updated.

In another embodiment, the control is a TRAF1 low control from a cell line of a similar type of tumour that has been treated with an shRNA to PKN1 (shPKN1) or to TRAF1 (shTRAF1).

In an embodiment, determining whether the cancer is TRAF1 low or TRAF1 high comprises measuring the expression of TRAF1 from a sample. In one embodiment, the sample is peripheral blood mononuclear cells or whole blood samples. In another embodiment, the sample is from a biopsy, such as from a diffuse large B cell lymphoma.

In another embodiment, determining whether the cancer is TRAF1 low or TRAF1 high further comprises assaying the expression level of TRAF1 in Raji control cells or Daudi control cells at the same time as measuring the level of TRAF1 in the sample.

In one embodiment, the nutrient stress-inducing agent is an asparaginase, arginase or glutaminase. In a particular embodiment, the nutrient stress inducing agent is Erwinase.

In an embodiment, the lymphocyte related cancer is of B cell origin. In another embodiment, the lymphocyte related cancer is of T cell origin. In yet another embodiment, the lymphocyte related cancer is lymphoma, such as Burkitt's lymphoma. In a further embodiment, the lymphocyte related cancer is leukemia, such as chronic lymphocytic leukemia (CLL).

Also provided herein is a method of determining treatment for lymphoma in a subject comprising:

a) determining whether the lymphoma is TRAF1 low or TRAF1 high compared to the level of expression in a control, optionally relative to RAJI or Daudi shPKN1 (TRAF1 low) or control RAJI or Daudi (TRAF1 high);

b) treating the subject with a nutrient stress-inducing agent if the subject is TRAF1 low; and

c) treating the subject with a nutrient stress-inducing agent in combination with an agent that lowers the levels of or inhibits TRAF1 if the subject is TRAF1 high.

Further provided herein is a method of determining treatment for leukemia in a subject comprising:

a) determining whether the leukemia is TRAF1 low or TRAF1 high compared to the level of expression in a control, optionally relative to Raji or Daudi shPKN1 (TRAF1 low) or control Raji or Daudi (TRAF1 high);

b) treating the subject with a nutrient stress-inducing agent if the subject is TRAF1 low; and

c) treating the subject with a nutrient stress-inducing agent in combination with an agent that lowers the levels of or inhibits TRAF1 if the subject is TRAF1 high.

Even further provided is an in vitro assay to identify an agent that induces the loss of TRAF1 activity comprising:

a) incubating cells with a nutrient stress inducing agent;

b) incubating cells with the nutrient stress inducing agent and a test compound;

c) incubating cells with the nutrient stress inducing agent and an shRNA to PKN1 or TRAF1;

d) incubating cells with the nutrient stress inducing agent, the shRNA to PKN1 or an shRNA to TRAF1 and the test compound; and

e) determining the level of cell survival in (a), (b), (c) and (d);

wherein a decreased level of cell survival in (b) as compared to (a), but not in (d) as compared to (c), indicates that the test compound inhibits TRAF1 activity directly or via inhibition of PKN1 activity.

Also provided is a method of treating a lymphocyte related cancer comprising administering a nutrient stress-inducing agent in combination with an agent that locally lowers the levels of or inhibits TRAF2. In an embodiment, the lymphocyte related cancer is of B-cell origin. In another embodiment, the lymphocyte related cancer is of T-cell origin. In another embodiment, the lymphocyte related cancer is lymphoma, such as Burkitt's lymphoma, or leukemia, such as chronic lymphocytic leukemia (CLL).

In another embodiment, the nutrient stress-inducing agent is asparaginase or arginase. In a particular embodiment, the asparaginase is Erwinase.

The agent that locally lowers the levels of or inhibits TRAF2 can inhibit TRAF2 expression or activity directly at the targeted site. In one embodiment, the agent that lowers the levels of or inhibits TRAF2 is an antisense or shRNA to TRAF2. In a particular embodiment, the shRNA is as shown in SEQ ID NO:4 or a variant thereof.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the role of TRAF1 and TRAF2 in signalling downstream of TNFR family members such as CD40 and 4-1 BB. (A) TRAF1 and TRAF2 are recruited to TNFR cytoplasmic tails and serve as adaptors to recruit cellular inhibitor of apoptosis, leading to downstream NF-κB activation and survival signalling. (B) The crystal structure of the coiled coil domain of TRAF1 and TRAF2, shows that they spontaneously form a heterotrimer that asymmetrically recruits one cellular inhibitor of apoptosis (cIAP) molecule.³ (C) Schematic of the domain structure of TRAF proteins.

FIG. 2 shows TRAF1 is essential to stabilize TRAF2 against degradation during nutrient stress following 4-1 BB signaling in primary T cells. WT and TRAF1^{-/-} OT-I (TCR transgenic) splenocytes were stimulated with their antigen (SIINFEKL peptide SEQ ID NO:3) for 48 hrs and rested for 24 hours. Activated OT-I CD8 T cells were isolated and stimulated in the presence of an isotype control or agonistic anti-4-1 BB antibody for 3 hours in RPMI 1640 media containing the indicated concentration of glutamine. TRAF2 and actin expression was determined by western blotting. Data are representative of at least two experiments.

FIG. 3 shows TRAF1 expression in human cancers. Left: RAJI cells were stably transfected with control small hairpin (sh) RNA or with shRNA designed to knockdown expression of TRAF1 (shRNAs obtained from Open Biosystems (TRC library number: TRCN0000056885 sequence: CGTGTGTTTGAGAACATTG) (SEQ ID NO:1)). TRAF1 levels in RAJI expressing shCtrl or with shTRAF1 were analyzed by Western blot. Protein loading was analyzed based on GAPDH levels. Right: TRAF1 levels in B-chronic lymphocytic leukemia (CLL) lines and Daudi CD5+ (a Burkitt's lymphoma with CLL like properties) were determined by Western blot.

FIG. 4 shows TRAF1 stabilizes TRAF2 against nutrient stress in lymphoma. shCtrl or shTRAF1 RAJI cells (from FIG. 3) were incubated in (A) media lacking glutamine (0 mM) or containing 1 mM glutamine, and immunoblotted for TRAF2. (B) Wild type (WT), shCtrl or shTRAF1 RAJI cells were treated with 1 IU/ml Erwinase for 1, 3 or 6 hrs and immunoblotted for TRAF2 and TRAF1.

FIG. 5 shows knockdown of PKN1 decreases TRAF1 levels in lymphoma. Left: Schematic diagram of TRAF1 indicating site of phosphorylation by PKN1 as identified by Kato et al.²⁹ Middle: RAJI cells were stably transfected with control shRNA (shCtrl), TRAF1 shRNA (shTRAF1) or PKN1 shRNA (shPKN1) (obtained from Open Biosystems (TRC library number: TRCN0000001484 sequence: CTGATGTGTGAGAAGCGGA (SEQ ID NO:2)). Right: Daudi cells were transduced with control shRNA (shCTRL), shRNA targeting TRAF1 (shTRAF1) or shRNA targeting PKN1 (shPKN1). Cells were analyzed for levels of TRAF1, tubulin or GAPDH by western blot. “Pool” refers to bulk transfected cell lines without subcloning.

FIG. 6 shows knockdown of PKN1 sensitizes RAJI cells to TRAF2 loss during nutrient stress. Left: Same figure as shown in FIG. 5, verifying knockdown of TRAF1 and PKN1. Right: RAJI cells stably expressing shCtrl or shPKN1 were treated with 1 IU/ml Erwinase for 0, 3 or 6 hrs and lysates were immunoblotted for TRAF2, or as a loading control, GAPDH.

FIG. 7 shows TRAF1 loss by direct knockdown or indirectly via PKN1 knockdown sensitizes lymphoma to growth inhibition by Erwinase. shCtrl, shTRAF1 and shPKN1 RAJI cells were treated with Erwinase as in FIG. 4 for the indicated times, before addition of MTT (3-(4,5-Dimethylthiazol-2-yl)-2,5-Diphenyltetrazolium Bromide) to assess cell viability. MTT reagent was then solubilized with DMSO and absorbance was measured by ELISA plate reader at 560 nm and referenced to absorbance at 620 nm. Data are plotted as fold change relative to untreated shCtrl RAJI cells.

FIG. 8 shows decreased TRAF1 combined with nutrient stress causes lymphoma death. RAJI cells stably expressing control shRNA (shCtrl), TRAF1 shRNA (shTRAF1) or PKN1 shRNA (shPKN1) were left untreated or treated with 1 IU/ml Erwinase for the indicated time points. For cells treated with Erwinase for longer than 24 hrs, cell culture media was replaced every 24 hrs and 1 IU/ml Erwinase was added to ensure the continued absence of glutamine. Cells were then stained with Annexin V-FITC (ebioscience) and Propidium Iodide (PI) and then analyzed by flow cytometry (BD LSR Fortessa). The upper right quadrant (Q2) indicates the fraction dead cells.

FIG. 9 shows (A) decreased TRAF1 combined with nutrient stress causes lymphoma death. Summary of data from FIG. 8; (B) Decreasing TRAF1 levels with shTRAF1 or shPKN1 increases the sensitivity of the Chronic lymphocytic leukemia cell line MEC2 to Erwinase induced death. MEC2 cells were transfected with control shRNA (shCTRL), shTRAF1 or shPKN1 and then were left untreated or treated with 1 IU/ml Erwinase for 0, 18, 24 or 42 hrs. Cell death was measured by flow cytometry for Annexin V and propidium iodide (PI), where double staining for Annexin V and Propidium iodide (PI) is used to indicate cell death. The results show that lowering TRAF1 or lowering PKN1 enhances the sensitivity of MEC2 to death induced by treatment with the nutrient stress-inducing drug Erwinase.

FIG. 10 shows evidence that TRAF1 Serine 146 (S146) is important in preventing its degradation. A. TRAF1 S146 is important for protein stability. 293 cells were transfected with equal amounts of either WT or TRAF1S146A mutant form of TRAF1. Cells were treated with cycloheximide to block new protein synthesis. The results show that WT TRAF1 is stable, but that mutant TRAF1S146A is unstable. B. Knockdown of PKN1 abrogates the differences in expression between WT and S146A TRAF1.

FIG. 11 shows TRAF1 expression positively correlates with mTOR activity. A. Western blot showing levels of pS6 are lower when TRAF1 is knocked down in RAJI. Left: Levels of pS6K in RAJI control cells. Right: Levels of pS6K in RAJI shTRAF1. Upper panels show pS6K, lower panels show GAPDH to control for equal loading. B. Schematic showing link between nutrient stress, mTORC1 and pS6. C. Left: Representative flow cytometry analysis of TRAF1 and phospho-S6 expression in Raji shCTRL and Raji shTraf1 cells. The results show that when TRAF1 is knocked down (left panels), there is a concomitant decrease in level of pS6 (middle panel). FMO is fluorescence minus one control stain. Right: Correlation plot of TRAF1 versus phospho-S6 expression in lymphoma and CLL cell lines (gated on live cells). All flow cytometry is based on gating on live cells (using live/dead staining marker).

FIG. 12 is a schematic of signaling with (a) PKN1 and (b) PKN1 knockdown. B lymphomas can express several TNFRs, including CD40, BAFFR and/or the EBV encoded protein LMP1, and constitutively signal to activate NF-κB and mitogen activated protein kinases (MAPKs), such as ERK. A. In PKN1 sufficient cells, TRAF1 protein is stable. TRAF1 is an NF-κB induced gene and thus is overexpressed in tumors with constitutive NF-κB activation. In turn, TRAF1 is a positive regulator ERK and NF-κB activation providing feedback enhancement of the ERK and NF-κB signaling pathways to perpetuate survival signaling in the lymphomas. The level of TRAF1 positively correlates with the level of pS6, a surrogate measure of the level of activation of mTORC1, a key sensor of nutrient status of cells. TRAF1 is important for ERK activation downstream of TNFRs.^{5, 30, 31} ERK is known to phosphorylate and thereby inactivate TSC2³², an upstream negative regulator of mTORC1. Thus TRAF1-ERK-signaling increases mTORC1 activity (as measured by pS6 levels) and thereby increases the resistance of cells to nutrient stress. The constitutively expressed kinase PKN1 phosphorylates TRAF1 on serine 146. B. Inhibition or knockdown of PKN1, or prevention of TRAF1 phosphorylation by mutating TRAF1 S146 to A, results in unstable TRAF1, resulting in lower TRAF1 levels in the cell, which in turn leads to lower ERK activation, lower NF-κB and lower activation of mTORC1, ultimately resulting in increased sensitivity to nutrient stress.

FIG. 13 shows TRAF1 expression predicts Erwinase sensitivity in Traf1^{med-hi} lymphoma cell lines. Correlation plot of TRAF1 expression versus Erwinase sensitivity. Cell lines were treated with 1 IU/ml Erwinase for 24 hours and cell death was measured by AnnexinV/PI staining. Erwinase sensitivity is measured as % of cells AnnexinV⁺, PI⁺ and/or AnnexinV⁺/PI⁺ cells. Left: Erwinase sensitivity versus TRAF1 expression (measured by flow cytometry, gated on live cells) for a panel of lymphoma and the CLL line MEC2. Dashed line shows the cut-off for medium/hi TRAF1 expression. Right: For Lymphomas/CLL with medium/hi TRAF1 expression there is an inverse correlation between TRAF1 levels and Erwinase sensitivity. dMFI indicates difference in mean fluorescence intensity between negative control and TRAF1 stain.

FIG. 14 shows TRAF2 knockdown effect. (A) Whole cell extracts from RAJI cells treated with control shRNA (shCTRL) or TRAF2 shRNA (shTRAF2) were immunoblotted for TRAF2 or as a loading control, GAPDH. (B) Annexin V staining of RAJI after Erwinase treatment (1 IU/ml; 24 hrs) in shCTRL, shTRAF1 or shTRAF2 cells. (C) TRAF1, PKN1 or TRAF2 knockdown lowers TRAF1 levels and reduces constitutive Erk and NF-κB activation in RAJI.

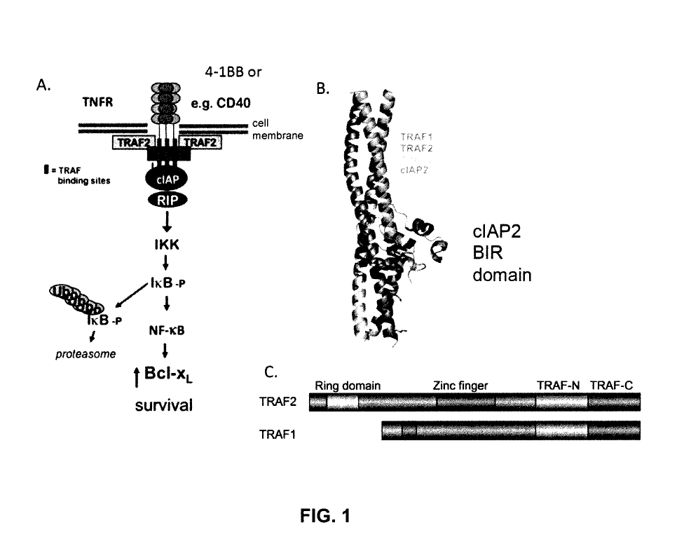

### DETAILED DESCRIPTION

The present inventors have demonstrated that lowering TRAF1 levels in Burkitt's lymphoma cells, directly or indirectly through PKN1 inhibition, under conditions of nutrient stress results in loss of TRAF2 and interferes with the constitutive survival signaling pathways in lymphomas and other TRAF1 positive cancers, causing their death. The present inventors have also shown sensitivity in chronic lymphocytic leukemia cells and have shown the importance of the serine 146 residue in TRAF1 on maintaining TRAF1 levels in cells.

Accordingly, the present disclosure provides a method of treating a lymphocyte related cancer in a subject in need thereof comprising administering a nutrient stress-inducing agent in combination with an agent that lowers the levels of or inhibits TRAF1. Also provided is use of a nutrient stress-inducing agent in combination with an agent that lowers the levels of or inhibits TRAF1 for treating a lymphocyte related cancer in a subject in need thereof. Further provided is use of a nutrient stress-inducing agent in combination with an agent that lowers the levels of or inhibits TRAF1 in the manufacture of a medicament for treating a lymphocyte related cancer in a subject in need thereof. Even further provided is a nutrient stress-inducing agent in combination with an agent that lowers the levels of or inhibits TRAF1 for use in treating a lymphocyte related cancer in a subject in need thereof.

In an embodiment, the lymphocyte related cancer is of B-cell origin. In another embodiment, the lymphocyte related cancer is of T-cell origin.

In one embodiment, the lymphocyte related cancer is lymphoma. In an embodiment, the lymphoma is Burkitt's lymphoma. In another embodiment, the lymphoma is B cell lineage non-Hodgkin's lymphoma. In yet another embodiment, the lymphoma is diffuse large B cell lymphoma.

In another embodiment, the lymphocyte related cancer is leukemia. In an embodiment, the lymphocyte related cancer is B-chronic lymphocytic leukemia (CLL).

In an embodiment, the subject has been identified as having a TRAF1 high expressing lymphocyte related cancer.

The term “nutrient stress-inducing agent” as used herein refers to an agent that inhibits protein synthesis and creates nutrient stress in the tumour microenvironment. In one embodiment, the nutrient stress-inducing agent reduces the levels of asparagine, arginine or glutamine. In an embodiment, the nutrient stress-inducing agent is an asparaginase or arginase. In a particular embodiment, the nutrient stress-inducing agent is Erwinase. Erwinaze (US), Erwinase (UK) is derived from the Asparaginase of Erwinia chrysanthemi. Its activity resides in asparaginase, an enzyme which degrades glutamine and arginine in the blood stream. It is FDA approved and prescribed for acute lymphoblastic leukemia (ALL) patients (http://www.fda.gov/NewsEvents/Newsroom/PressAnnouncements/ucm280525.htm).²⁵ The nutrient stress-inducing agent can also be any similar amino acid degrading enzyme that limits essential amino acid availability to the tumour.

The term “TRAF1” as used herein refers to TRAF1, or Tumour necrosis factor Receptor Associated Factor 1, from any species or source, optionally mammalian, such as human or mouse. For example, TRAF 1 has Genbank accession numbers: NM_005658, NM_001190945, NM_001190947.

The term “TRAF2” as used herein refers to TRAF2, or Tumour necrosis factor Receptor Associated Factor 2, from any species or source, optionally mammalian, such as human or mouse. For example, TRAF2 has a Genbank accession number: NM_021138.

The term “an agent that lowers the levels of or inhibits TRAF1” as used herein refers to any agent that is capable of reducing the levels or activity of TRAF1, either directly or indirectly, such that TRAF1 activity of stabilizing TRAF2 is reduced or inhibited. The “agent that lowers the levels of or inhibits TRAF1” includes any substance that is capable of inhibiting/reducing the expression or activity of TRAF1, in particular, its activity in stabilizing TRAF2, and thus, includes substances that inhibit TRAF1 expression, stability or activity. Such inhibitors optionally include antisense nucleic acid molecules, small interfering RNA molecules, proteins, antibodies (and fragments thereof) that are internalized or expressed within the cell, small molecule inhibitors, peptide based inhibitors and other substances.

In one embodiment, the agent that lowers the levels of or inhibits TRAF1 is an antisense or small interfering RNA molecule to TRAF1. In a particular embodiment, the agent that lowers the levels of or inhibits TRAF1 is an shRNA comprising the nucleotide sequence CGTGTGTTTGAGAACATTG (SEQ ID NO:1) or a variant thereof.

The present inventors have shown that reduction in PKN1 results in reduced levels or activity of TRAF1. Accordingly, in another embodiment, the agent that lowers the levels of or inhibits TRAF1 is an inhibitor of PKN1.

The term “PKN1” as used herein refers to PKN1, or Protein Kinase N-1, from any species or source, optionally mammalian, such as human or mouse. For example, PKN1 has Genbank accession numbers: NM_213560, NM_002741. PKN1 is also referred to in the literature as PAK1, PKN, PRK1 or PRKCL1.

The term “PKN1 inhibitor” as used herein includes any substance that is capable of inhibiting/reducing the expression or activity of PKN1, in particular, its activity in reducing TRAF1 levels and in particular, its kinase activity, and thus, includes substances that inhibit PKN1 expression or activity. Such inhibitors optionally include antisense nucleic acid molecules, small interfering RNA molecules, proteins, antibodies (and fragments thereof) that are internalized or expressed within the cell, small molecule inhibitors, peptide based inhibitors and other substances.

In one embodiment, the agent that lowers the levels of or inhibits TRAF1 is an antisense or shRNA to PKN1. In a particular embodiment, the shRNA to PKN1 comprises the nucleotide sequence CTGATGTGTGAGAAGCGGA (SEQ ID NO:2) or a variant thereof.

TRAF1 deficient mice are healthy and fertile whereas TRAF2 deficient mice die perinatally of inflammation. Thus, TRAF1 inhibition is a strategy for reducing TRAF2 in a limited manner. Furthermore, TRAF2, unlike TRAF1, is constitutively expressed in normal and cancer cells, which is one of the benefits of therapeutically inhibiting TRAF1's ability to stabilize TRAF2. However, TRAF2 knockdown if targeted to the tumour also will increase sensitivity to Erwinase or another nutrient stress inducing agent.

Accordingly, also provided is a method of treating a lymphocyte related cancer comprising administering a nutrient stress inducing agent in combination with an agent that locally lowers the levels of or inhibits TRAF2. Even further provided is a use of a nutrient stress-inducing agent in combination with an agent that lowers the levels of or inhibits TRAF2 for treating a lymphocyte related cancer. Also provided is a use of a nutrient stress-inducing agent in combination with an agent that lowers the levels of or inhibits TRAF2 in the preparation of a medicament for treating a lymphocyte related cancer. Further provided is a nutrient stress-inducing agent in combination with an agent that lowers the levels of or inhibits TRAF2 for use in treating a lymphocyte related cancer. In an embodiment, the lymphocyte related cancer is of B-cell origin. In another embodiment, the lymphocyte related cancer is of T-cell origin. In another embodiment, the lymphocyte related cancer is lymphoma, such as Burkitt's lymphoma, or leukemia, such as chronic lymphocytic leukemia (CLL).

In another embodiment, the nutrient stress-inducing agent is asparaginase or arginase. In a particular embodiment, the asparaginase is Erwinase.

The agent that locally lowers the levels of or inhibits TRAF2 can inhibit TRAF2 expression or activity directly at the targeted site. In one embodiment, the agent that lowers the levels of or inhibits TRAF2 is an antisense or shRNA to TRAF2. In a particular embodiment, the shRNA is as shown in SEQ ID NO: 4 or a variant thereof.

In an embodiment, the subject has been identified as having a TRAF1 high expressing lymphocyte related cancer.

The term “antisense nucleic acid” as used herein means a nucleic acid that is produced from a sequence that is inverted relative to its normal presentation for transcription. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The terms “RNA interference,” “interfering RNA” or “RNAi” refer to single-stranded RNA or double-stranded RNA (dsRNA) that is capable of reducing or inhibiting expression of a target nucleic acid by mediating the degradation of mRNAs which are complementary to the sequence of the interfering RNA when the interfering RNA is in the same cell as the target gene. Interfering RNA may have substantial or complete identity to the target nucleic acid or may comprise a region of mismatch.

The term “siRNA” or “siRNA oligonucleotide” refers to a short inhibitory RNA that can be used to reduce or inhibit nucleic acid expression of a specific nucleic acid by RNA interference.

The siRNA can be a duplex, a short RNA hairpin (shRNA) or a microRNA (miRNA).

A person skilled in the art will recognize that an RNA molecule can be altered by substituting uracil (U) with thymine (T) residues without abolishing the ability of the resulting molecule to inhibit RNA expression. Optionally, the nucleic acids disclosed herein are chemically synthesized.

Methods of designing specific nucleic acid molecules that silence gene expression and administering them are known to a person skilled in the art. For example, it is known in the art that efficient silencing is obtained with siRNA duplex complexes paired to have a two nucleotide 3′ overhang. The siRNA can also be chemically modified to increase stability. For example adding two thymidine nucleotides and/or 2′O methylation is thought to add nuclease resistance. Other modifications include the addition of a 2′-O-methyoxyethyl, 2′-O-benzyl, 2′-O-methyl-4-pyridine, C-allyl, O-allyl, O-alkyl, O-alkylthioalkyl, O-alkoxylalkyl, alkyl, alkylhalo, O-alkylhalo, F, NH2, ONH2, O-silylalkyl, or N-phthaloyl group (see U.S. Pat. No. 7,205,399; Kenski et al. Mol. Ther. Nucl. Acids 1:1-8 (2012); Behlke, Oligonucleotides 18:305-320 (2008)). Other modifications include direct modification of the internucleotide phosphate linkage, for example replacement of a non-bridging oxygen with sulfur, boron (boranophosphate), nitrogen (phosphoramidate) or methyl (methylphosphonate). A person skilled in the art will recognize that other nucleotides can also be added and other modifications can be made. As another example deoxynucleotide residues (e.g. dT) can be employed at the 3′ overhang position to increase stability.

The term “miRNA” refers to microRNAs which are small non-coding RNA molecules, for example 22 nucleotides, that are processed from hairpin RNA precursors, for example about 70 nucleotides long. miRNAs can inhibit gene expression through targeting homologous mRNAs.

In another embodiment, the agent that lowers the levels of or inhibits TRAF1 is an antibody that binds to and inhibits TRAF1 or PKN1 or a mimetic or aptamer provided that the agent is internalized or expressed within the cell.

Aptamers are short strands of nucleic acids that can adopt highly specific 3-dimensional conformations. Aptamers can exhibit high binding affinity and specificity to a target molecule. These properties allow such molecules to specifically inhibit the functional activity of proteins. Thus, in another embodiment, the agent that lowers TRAF1 is an aptamer that binds and inhibits TRAF1 or PKN1.

The term “antibody” as used herein is intended to include fragments thereof which also specifically react with a TRAF1 or PKN1. Antibodies can be fragmented using conventional techniques and the fragments screened for utility in the same manner as described below. For example, F(ab′)2 fragments can be generated by treating antibody with pepsin. The resulting F(ab′)₂ fragment can be treated to reduce disulfide bridges to produce Fab′ fragments.

Conventional methods can be used to prepare antibodies. For example, by using a TRAF1 or PKN1 peptide, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)) and screening of combinatorial antibody libraries (Huse et al., Science 246:1275 (1989)). Hybridoma cells can be screened immunochemically for production of antibodies specifically reactive with the peptide and the monoclonal antibodies can be isolated. Therefore, the disclosure also contemplates hybridoma cells secreting monoclonal antibodies with specificity for a TRAF1 or PKN1.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes a TRAF1 or PKN1 protein (See, for example, Morrison et al. (PNAS 81:21 6851-6855, 1984), and Takeda et al. (Nature 314:452-454), and the patents of Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a TRAF1 or PKN1 as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al. (PNAS 80:12 7308-7312, 1983), Kozbor et al. (Immunology Today 4:3 72-79, 1983); Olsson et al. (Methods in Enzymol, 92:3-16 1982) and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against a TRAF1 or PKN1 may also be generated by screening expression libraries encoding immunoglobulin genes, or portions thereof, expressed in bacteria with peptides produced from the nucleic acid molecules encoding a somatostatin or somatostatin receptor. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)).

Antibodies may also be prepared using DNA immunization. For example, an expression vector containing a nucleic acid encoding a TRAF1 or PKN1 may be injected into a suitable animal such as mouse. The protein will therefore be expressed in vivo and antibodies will be induced. The antibodies can be isolated and prepared as described above for protein immunization.

The TRAF1 or PKN1 inhibitors may also contain or be used to obtain or design “peptide mimetics”. For example, a peptide mimetic may be made to mimic the function of a TRAF1 or PKN1 antibody. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of the protein, including biological activity. Peptide mimetics also include peptoids, oligopeptoids (Simon et al ((1992) PNAS 89:9367-9371)).

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins of the invention. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term a “therapeutically effective amount”, “effective amount” or a “sufficient amount” of a compound of the present application is a quantity sufficient to, when administered to the subject, including a mammal, for example a human, effect beneficial or desired results, including clinical results, and, as such, an “effective amount” or synonym thereto depends upon the context in which it is being applied. An “effective amount” is intended to mean that amount of an agent that is sufficient to treat, prevent or inhibit such diseases. The amount of a given agent that will correspond to such an amount will vary depending upon various factors, such as the given agent, the pharmaceutical formulation, the route of administration, the type of condition, disease or disorder, the identity of the subject or host being treated, and the like, but can nevertheless be routinely determined by one skilled in the art. Also, as used herein, a “therapeutically effective amount” of an agent is an amount which prevents, inhibits, suppresses or reduces a disorder, disease or conditions that benefits from the agents, as determined by clinical symptoms, in a subject as compared to a control. As defined herein, a therapeutically effective amount of an agent may be readily determined by one of ordinary skill by routine methods known in the art.

Moreover, a “treatment” or “prevention” regime of a subject with a therapeutically effective amount of an agent may consist of a single administration, or alternatively comprise a series of applications. For example, the agent may be administered at least once a week. However, in another embodiment, the agent may be administered to the subject from about one time per week to about once daily for a given treatment. The length of the treatment period depends on a variety of factors, such as the severity of the disease, the age of the patient, the concentration and the activity of the agent, or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required.

The term “administering” is defined as any conventional route for administering an agent(s) to a subject for use as is known to one skilled in the art. This may include, for example, administration via the parenteral (i.e. subcutaneous, intradermal, intramuscular, etc.) or mucosal surface route. In other embodiments this may include oral administration. The dose of the agent(s) may vary according to factors such as the health, age, weight and sex of the animal. The dosage regime may be adjusted to provide the optimum dose. One skilled in the art will appreciate that the dosage regime can be determined and/or optimized without undue experimentation.

To “inhibit” or “suppress” or “lower” or “reduce” or “downregulate” a function or activity, such as TRAF1 expression or activity, is to reduce the function or activity when compared to otherwise same conditions except for a condition or parameter of interest, or alternatively, as compared to another condition or control.

The agents, the agent that lowers the levels of or inhibits TRAF1 and the nutrient stress-inducing agent, disclosed herein may be administered contemporaneously. As used herein, “contemporaneous administration” of two substances to an individual means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances.

In one embodiment, the subject is an animal. The term “animal” includes all members of the animal kingdom, optionally a mammal, such as a human.

The term “TRAF1 low” as used herein refers to a level of expression of TRAF1 indicative of sensitivity to nutrient stress. The term “TRAF1 high” as used herein refers to a level of expression of TRAF1 indicative of an increase in sensitivity to nutrient stress in the presence of an agent that lowers the levels of or inhibits TRAF1 compared to the absence of the agent. The present disclosure defines a range of TRAF1 levels which result in insensitivity or sensitivity to nutrient stress in RAJI cells and Daudi CD5⁺ cells. The untreated or shCtrl cells have a level of TRAF1 which renders them insensitive to Erwinase, whereas cells in which TRAF1 levels have been lowered using PKN1 knockdown or TRAF1 knockdown (shTRAF1 or shPKN1 RAJI) provide a starting point to define low TRAF1 expression as these cells are Erwinase sensitive, i.e. TRAF1 low. Therefore, using RAJI/Daudi shCntrl and RAJI/Daudi shPKN1 as a starting point to define TRAF1 high and low, a larger panel of lymphocyte related cancers can be examined with graded TRAF1 expression to refine the cutoffs for TRAF1 levels and Erwinase sensitivity and this information can be used to stratify primary patient lymphomas/leukemias based on TRAF1 levels for their potential Erwinase sensitivity. It is recognized that the level of TRAF1 that defines nutrient stress sensitivity may depend on the type of lymphoma or CLL. Thus calibration will be done for each cancer subtype.

Accordingly, herein provided is a method of stratifying lymphocyte related cancers comprising developing a standard curve for a class of tumour (such as Burkitts lymphoma, B-cell lineage Hodgkins lymphoma, CLL or diffuse large B cell lymphoma, DLBCL) by comparing TRAF1 expression with sensitivity to Erwinase on the samples; determining a cutoff value of expression for TRAF1 levels and Erwinase resistance; and identifying the tumour as either a high TRAF1 or low TRAF1.

In one embodiment, samples may be frozen immediately after collection. Samples may be thawed, rested overnight and then left untreated or treated with 1 U/ml Erwinase in vitro for 1, 2 or 3 days. At each time point percent survival will be measured and compared with the TRAF1 level. A cutoff for TRAF1 levels and Erwinase resistance will be determined in order to identify those tumors with a sufficiently high TRAF1 dMFI that will likely benefit from PKN1 or TRAF1 inhibition. Once this is established for a panel of banked class of tumour samples, this assay will be used to test prospective patients using a sample, such as peripheral blood mononuclear cell or whole blood samples from for example, CLL patients or biopsies from, for example, DLBCL patients. To put TRAF1 levels on a scale that can be transferred from assay to assay, each assay may include RAJI/Daudi cntrl and RAJI/Daudi shTRAF1 (to test specificity of stain) and the level of TRAF1 (TRAF1 dMFI) in the primary sample relative to RAJI/Daudi CTRL will be compared so that a level relative to the RAJI/Daudi standard cell line can be obtained.

In one embodiment, the present disclosure provides a method of determining treatment for a lymphocyte related cancer in a subject comprising:

a) determining whether the lymphocyte related cancer is TRAF1 low or TRAF1 high compared to a control;

b) treating the subject with a nutrient stress-inducing agent if the lymphocyte related cancer is TRAF1 low; and

c) treating the subject with a nutrient stress-inducing agent in combination with a an agent that lowers the levels of or inhibits TRAF1 if the lymphocyte related cancer is TRAF1 high.

In another embodiment, the present disclosure provides a method of determining treatment for a lymphocyte related cancer in a subject comprising determining whether the lymphocyte related cancer is TRAF1 low or TRAF1 high; wherein if the cancer is TRAF1 low it is indicative of treating the cancer with a nutrient stress-inducing agent and if the cancer is TRAF1 high it is indicative of treating the cancer with a nutrient stress-inducing agent in combination with an agent that lowers the levels of or inhibits TRAF1.

In one embodiment, the control is a TRAF1 high control from the same type of tumour or from a pool of positive tumours of the same type.

In yet another embodiment, the TRAF1 high control is a reference standard or standard curve, optionally obtained from a panel of banked samples and optionally determined relative to Raji or Daudi cells. In an embodiment, the reference standard is from historical data for a pool of patients with the same class of tumour and optionally this data is continually updated.

In another embodiment, the control is a TRAF1 low control from a cell line of a similar type of tumour that has been treated with shPKN1 or shTRAF1 to reach a level that renders the cell sensitive to nutrient stress induced death alone.

In an embodiment, determining whether the cancer is TRAF1 low or TRAF1 high comprises measuring the expression of TRAF1 from a sample.

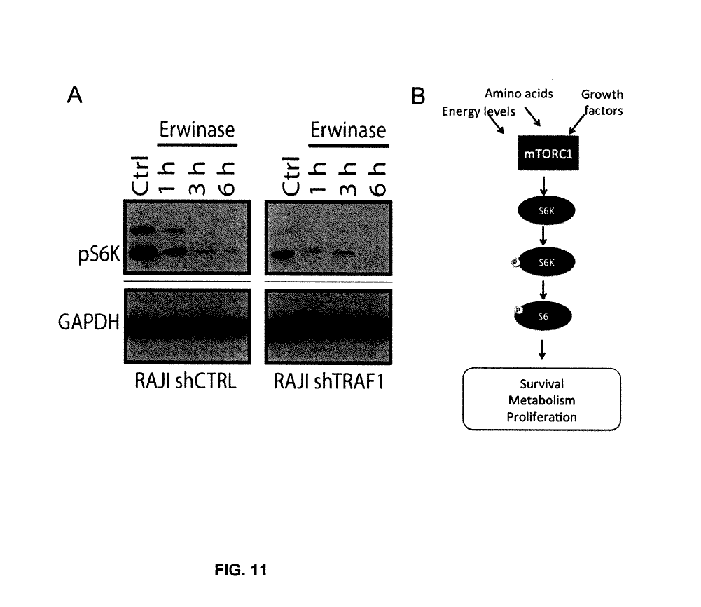

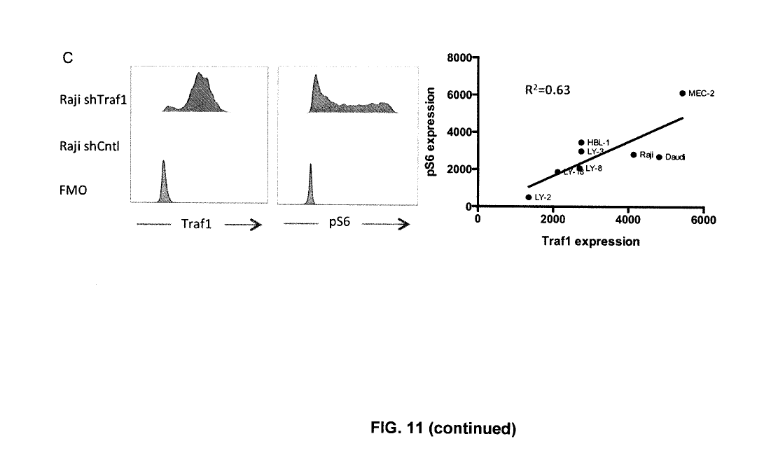

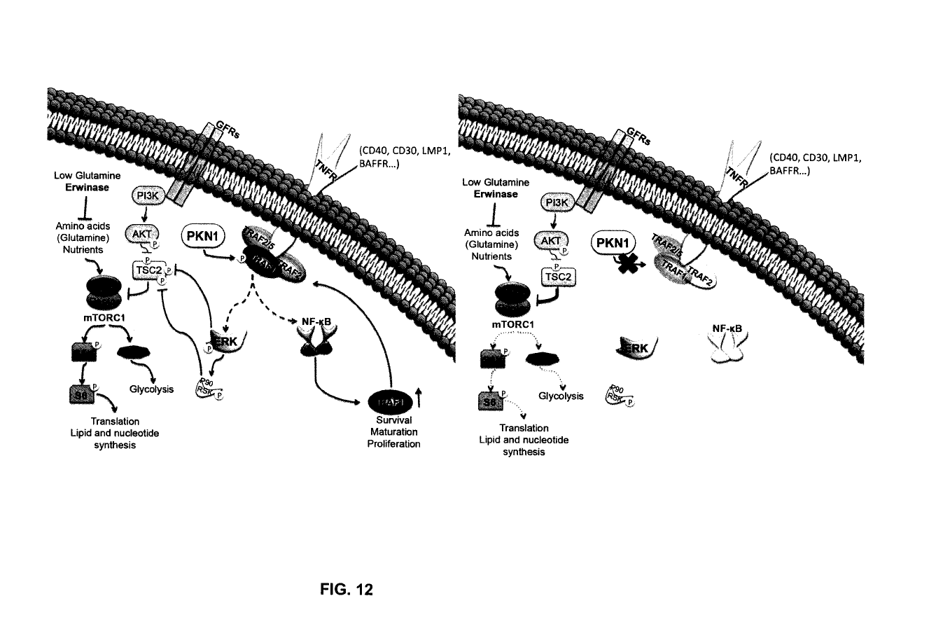

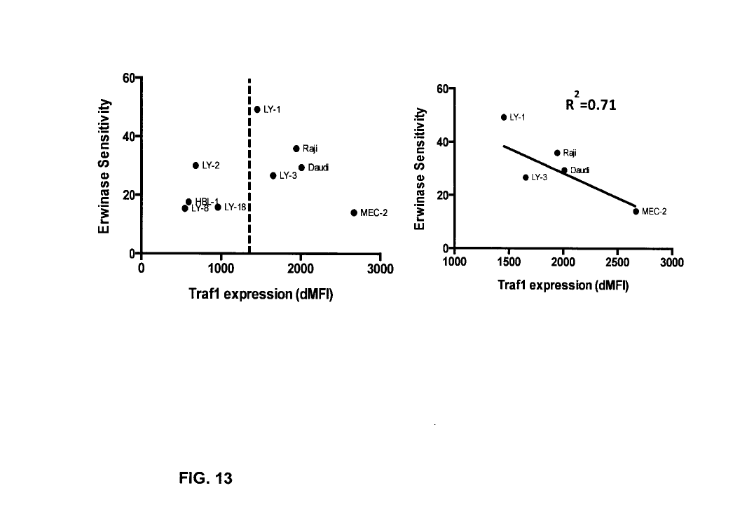

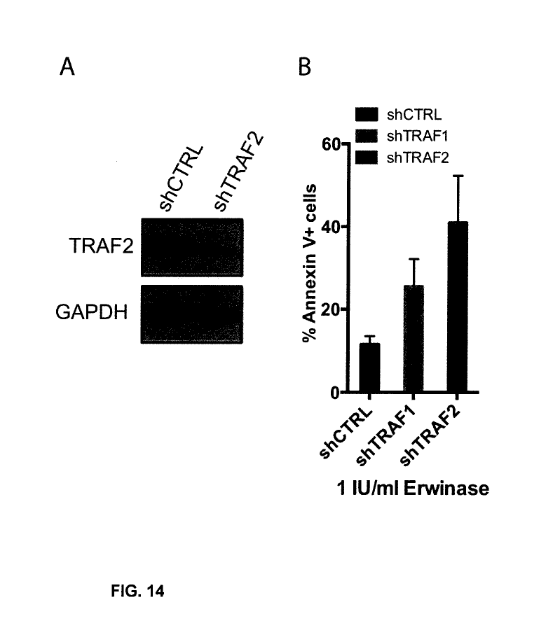

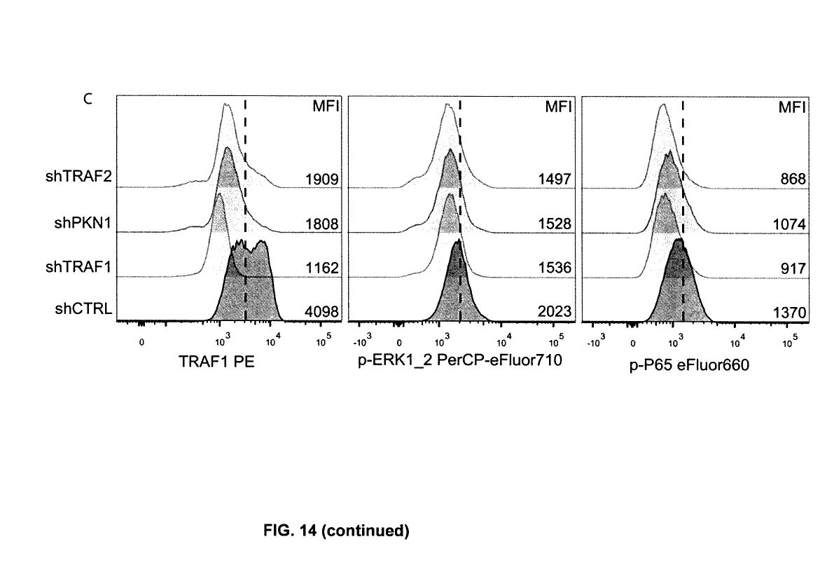

The level of TRAF1 in the sample may be measured by known techniques, including without limitation, flow cytometry after permeabilization of the cells, for example as shown in FIGS. 13 and 14C or by western blot on whole cell lysates, for example, as shown in FIG. 11A, or, as TRAF1 is known to be transcriptionally regulated, by determining the amount of TRAF1 mRNA using quantitative PCR, for example, on RNA isolated from PBMC or patient biopsies.

Accordingly, in one embodiment, the sample is peripheral blood mononuclear cells or whole blood samples. In another embodiment, the sample is from a biopsy, such as from a diffuse large B cell lymphoma.

In another embodiment, determining whether the cancer is TRAF1 low or TRAF1 high further comprises assaying the expression level in Raji control cells or Daudi control cells at the same time as measuring the level of TRAF1 in the sample.

In one embodiment, the nutrient stress-inducing agent is an asparaginase, arginase or glutaminase. In a particular embodiment, the nutrient stress inducing agent is Erwinase.

In an embodiment, the lymphocyte related cancer is of B cell origin. In another embodiment, the lymphocyte related cancer is of T cell origin. In yet another embodiment, the lymphocyte related cancer is lymphoma, such as Burkitt's lymphoma, B-cell non-Hodgkins lymphoma or Diffuse B cell Lymphoma (D-LBCL). In a further embodiment, the lymphocyte related cancer is leukemia, such as chronic lymphocytic leukemia (CLL).

In a particular embodiment, the present disclosure provides a method of determining treatment for lymphoma in a subject comprising:

a) determining whether the lymphoma is TRAF1 low or TRAF1 high compared to the level of expression of a control, optionally relative to RAJI or Daudi shPKN1 (TRAF1 low) or control RAJI (TRAF1 high);

b) treating the subject with a nutrient-stress inducing agent if the subject is TRAF1 low; and

c) treating the subject with a nutrient-stress inducing agent in combination with an agent that lowers the levels of or inhibits TRAF1 if the subject is TRAF1 high.

In another particular embodiment, the present disclosure provides a method of determining treatment for leukemia in a subject comprising:

a) determining whether the leukemia is TRAF1 low or TRAF1 high compared to the level of expression of a control, optionally relative to Raji or Daudi shPKN1 (TRAF1 low) or control Raji (TRAF1 high);

b) treating the subject with a nutrient stress-inducing agent if the subject is TRAF1 low; and

c) treating the subject with a nutrient stress-inducing agent in combination with an agent that lowers the levels of or inhibits TRAF1 if the subject is TRAF1 high.

The term “isolated” as used herein refers to a nucleic acid substantially free of cellular material or culture medium when produced by recombinant DNA techniques, or chemical precursors, or other chemicals when chemically synthesized.

The isolated nucleic acid molecule that silences the expression of TRAF1 or PKN1, such as siRNA, shRNA or antisense molecule, is optionally chemically modified to increase stability as disclosed herein.

Nucleic acids of the disclosure also include variant nucleic acids that comprise at least 70%, 80%, 90%, 95%, 98%, 99% or 100% nucleic acid sequence identity with the nucleic acid molecules of the disclosure that retain inhibition, e.g. nucleic acid molecules that silence the expression of TRAF1 or PKN1, when transcribed. For example, the variant nucleic acid in one embodiment comprises a nucleic acid sequence that is at least 70%, 80%, 90%, 95%, 98%, 99% or 100% identical to a nucleic acid sequence selected from SEQ ID NO:1, 2 or 4. Such variant nucleic acid sequences include nucleotide sequences that hybridize to the nucleic acids corresponding to SEQ ID NOs: 1, 2 or 4 under at least moderately stringent hybridization conditions.

To determine the percent identity of two sequences, the sequences are aligned for optimal comparison purposes (e.g., gaps can be introduced in the sequence of a first amino acid or nucleic acid sequence for optimal alignment with a second amino acid or nucleic acid sequence). The amino acid residues at corresponding amino acid positions are then compared in the case of a protein sequence. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecule of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

Nucleic acid molecules of the disclosure can be constructed using chemical synthesis and enzymatic ligation reactions using procedures known in the art. For example, nucleic acid molecules that silence the expression of TRAF1 or PKN1 can be chemically synthesized using naturally occurring nucleotides or variously modified nucleotides designed to increase the biological stability of the molecules or to increase the physical stability of the duplex formed between the antisense and sense nucleic acids in the case of the siRNA, e.g., phosphorothioate derivatives and acridine-substituted nucleotides can be used.

Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine. Alternatively, the nucleic acid molecules can be produced biologically using an expression vector.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complementary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

The agents for use in the methods and uses of the disclosure are suitably formulated into pharmaceutical compositions for administration to subjects, for example human subjects, in a biologically compatible form suitable for administration in vivo. Accordingly, also provided herein is a pharmaceutical composition comprising an isolated nucleic acid molecule that silences the expression of TRAF1 or PKN1 disclosed herein and a pharmaceutically acceptable carrier or diluent. Also provided is a pharmaceutical composition comprising an agent that lowers the levels of or inhibits TRAF1 as disclosed herein and a nutrient stress inducing agent as disclosed herein and optionally, a pharmaceutically acceptable carrier or diluent.

The compositions containing the agents can be prepared by known methods for the preparation of pharmaceutically acceptable compositions which can be administered to subjects, such that an effective quantity of the active agent is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the agents in association with one or more pharmaceutically acceptable vehicles or diluents, and contained in buffered solutions with a suitable pH and iso-osmotic with the physiological fluids.

The agents may be administered to a subject alone or in combination with pharmaceutically acceptable carriers, as noted above, and/or with other pharmaceutically active agents for the treatment of the lymphocyte related cancer, the proportion of which is determined by the solubility and chemical nature of the agents, chosen route of administration and standard pharmaceutical practice.

The dosage of the agents and/or compositions can vary depending on many factors such as the pharmacodynamic properties of the agent, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The agents may be administered initially in a suitable dosage that may be adjusted as required, depending on the clinical response. For ex vivo treatment of cells over a short period, for example for 30 minutes to 1 hour or longer, higher doses of agent may be used than for long term in vivo therapy.

### Assays

There are currently no specific PKN1 inhibitors in the public domain. Lestauritinib³³ can inhibit PKN1 in the nm range but this inhibitor is non-specific and inhibits other kinases such as FLT3³³ and JAK2³⁴ with at least as low an IC50. Similarly, pan-PKC inhibitors would not be suitable, due to broad inhibitory effects, which increases the toxicity and potential for side effects. Thus the present studies identify PKN1 as a specific target for drug discovery, for which a suitable chemical inhibitor has not yet been identified.

Accordingly, the present disclosure provides an in vitro assay to identify a PKN1 inhibitor useful for treating lymphocyte related cancers comprising:

a) incubating cells with a nutrient stress-inducing agent;

b) incubating cells with the nutrient stress-inducing agent and a test compound;

c) incubating cells with the nutrient stress-inducing agent and an shRNA to PKN1 or TRAF1;

d) incubating cells with the nutrient stress-inducing agent, the shRNA to PKN1 or TRAF1, and the test compound; and

e) determining the level of cell survival in (a), (b), (c) and (d);

wherein a decreased level of cell survival in (b) as compared to (a), but not in (d) as compared to (c), indicates that the test compound inhibits TRAF1 activity directly or via inhibition of PKN1 activity.

In one embodiment, the nutrient stress-inducing agent is an asparaginase or arginase. In a particular embodiment, the nutrient stress inducing agent is Erwinase.

In an embodiment, the cells are a Raji Burkitt's cell line or a diffuse large B cell lymphoma line or other suitable lymphoma line. In another embodiment, the cells are Daudi CD5⁺ cells. A person skilled in the art would readily be able to ascertain other suitable cell lines that show additive effects of nutrient stress combined with TRAF1 or PKN1 knockdown.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### Examples

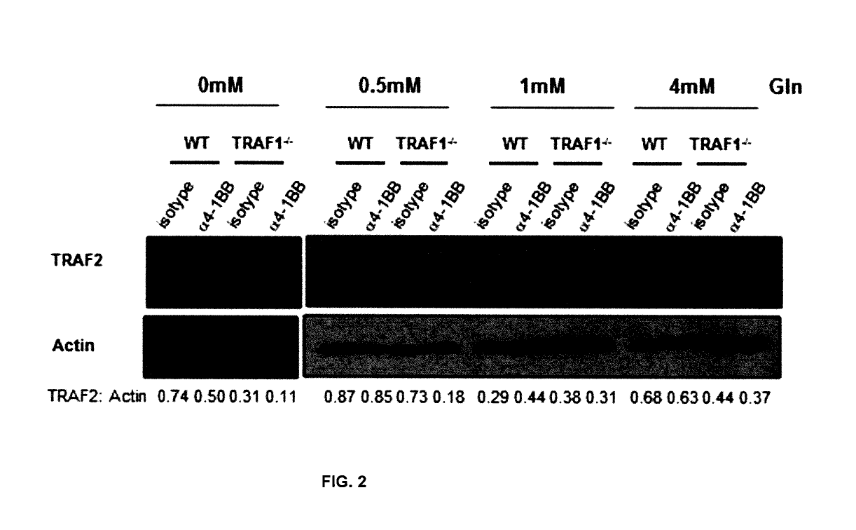

During the course of studies to investigate the mechanism of the loss of TRAF2 in the absence of TRAF1, it was noted that the loss of TRAF2 in the absence of TRAF1 depended on the level of nutrients available to the cell. When primary T cells were induced to proliferate in the presence of 4-1BB signalling, TRAF2 was degraded in TRAF1^{-/-} but not wild-type cells, but only when growth conditions were limiting, such as when glutamine or glucose levels were low in the culture medium (FIG. 2). In contrast, when glutamine levels were high, TRAF2 was relatively stable during 4-1BB induced signalling in proliferating T cells (FIG. 2). Further analysis, indicated that TRAF2 was degraded in a proteasome dependent manner and preliminary data indicate that this degradation involves the E3 ligase activity of a protein called siah2.

TRAF1 Knockdown in Lymphoma Leads to Loss of TRAF2 During Nutrient Stress.

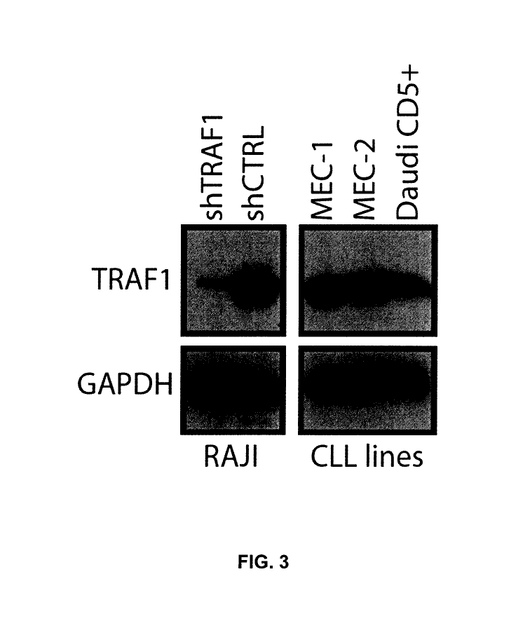

Based on the finding that primary T cells lacking TRAF1 are sensitive to loss of TRAF2 when 4-1 BB signalling is induced during nutrient stress, the role of the reduction of TRAF1 levels combined with nutrient stress on B lymphomas was tested. As proof of principal, the present inventors focused on the CD40-positive Burkitt's lymphoma, RAJI, which expresses high levels of TRAF1 (FIG. 3). A version of RAJI with low levels of TRAF1 was generated by transfecting RAJI with a construct expressing a small hairpin (sh) RNA targeting TRAF1 (shTRAF1) to generate a stable cell line with low TRAF1 expression (shTRAF1 RAJI) (CGTGTGTTTGAGAACATTG—SEQ ID NO:1). The resulting cell line had substantially lower levels of TRAF1 than the control treated RAJI (FIG. 3). Several CLL lines obtained from the Minden Lab, Ontario Cancer Institute were also screened and it was confirmed that these lymphomas also express TRAF1, consistent with the previous report that CLL express TRAF1¹⁰ (FIG. 3, right). Of note, normal B cells have undetectable levels of TRAF1.¹⁰

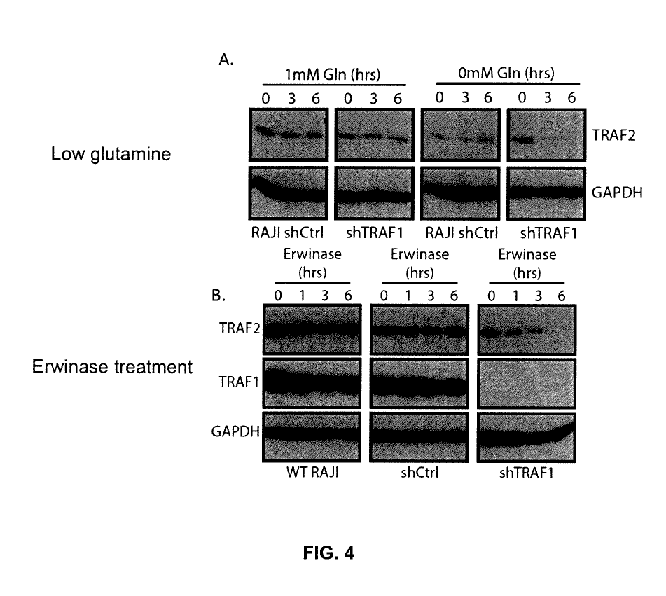

To test the effect of lower levels of TRAF1 on the level of TRAF2 in lymphoma, WT and shTRAF1 RAJI cells were incubated under conditions of nutrient stress (low glutamine). TRAF2 was lost in TRAF1 low but not WT cells (FIG. 4, top panels). In order to mimic conditions of nutrient stress in a manner that can be useful therapeutically, the licensed drug Erwinase (described above) was utilized. Treatment of RAJI cells with Erwinase resulted in a significant reduction in TRAF2 levels only when TRAF1 was reduced by shRNA (FIG. 4, lower panels). Of note, in contrast to the work with primary cells, the loss of TRAF2 when TRAF1 is limiting occurred without a need to add reagents to specifically engage a TNFR family member, but rather occurs spontaneously upon nutrient stress.

PKN1 Regulates TRAF1 Levels.

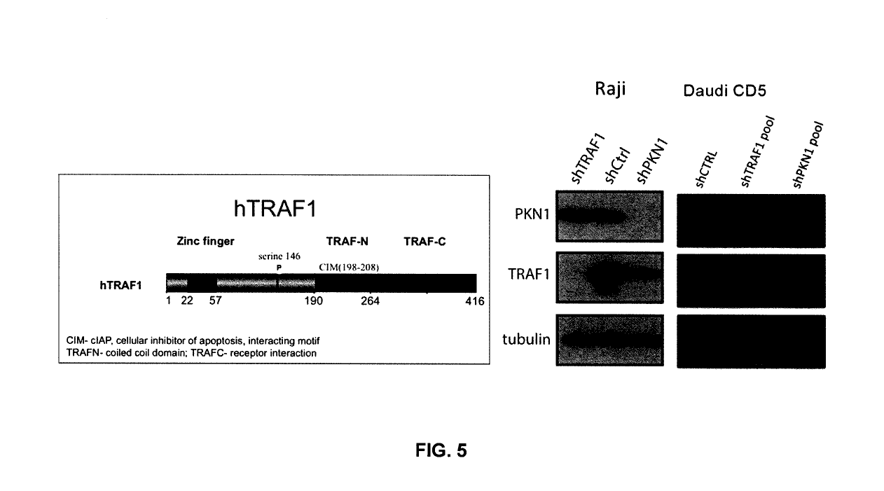

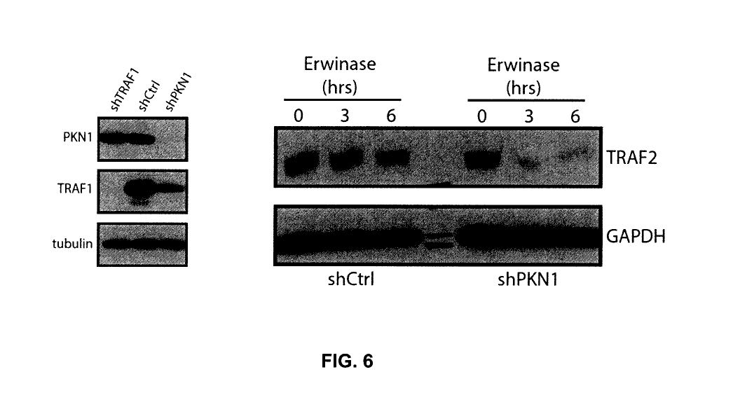

TRAF1 was previously reported to be a target of phosphorylation by the protein kinase C family member PKN1.²⁹ In the published work, it was suggested based on overexpression studies that PKN1-TRAF1 interaction negatively regulated the NF-κB pathway²⁹ and as such, lowering levels of PKN1 and TRAF1 would have been expected to increase lymphoma survival. However, the opposite result was found. RAJI lymphoma or Daudi CD5+ cells were transfected with an shRNA that reduces the level of PKN1 in the cells (CTGATGTGTGAGAAGCGGA—SEQ ID NO:2). The decreased expression of PKN1 resulted in lower levels of TRAF1 (FIG. 5), suggesting that PKN1 phosphorylates TRAF1 to stabilize it against degradation. Reducing PKN1 levels had a similar effect on inducing TRAF2 loss during nutrient stress (FIG. 6) as had been observed with TRAF1 knockdown (see FIG. 4).

Decreasing TRAF1 Levels with shTRAF1 or shPKN1 Increases the Sensitivity of RAJI to Erwinase Induced Death.

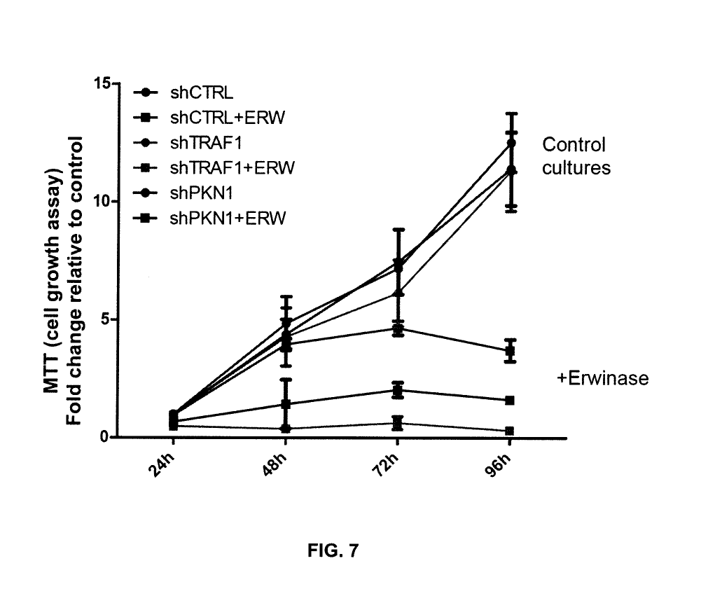

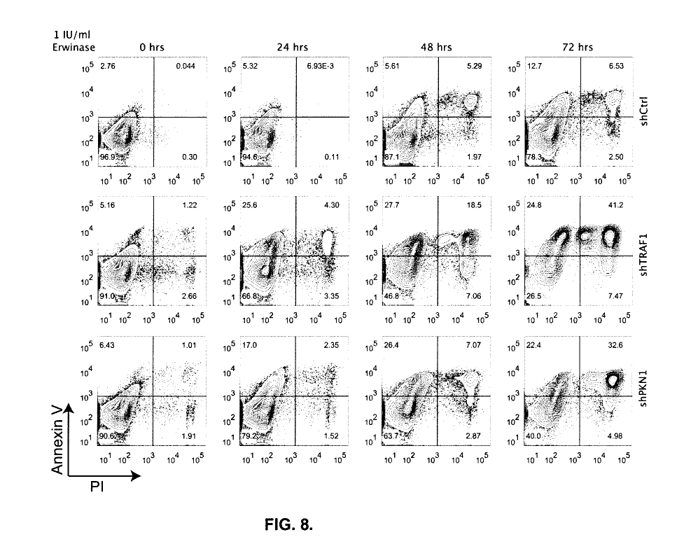

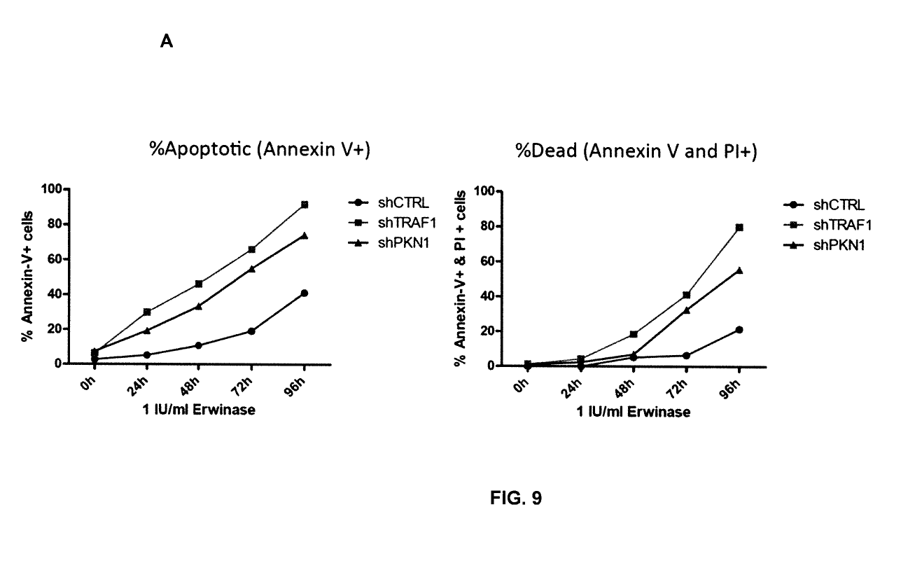

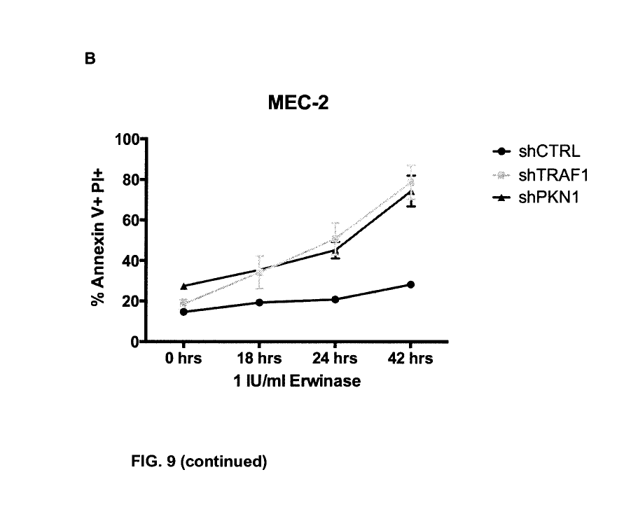

It was next asked what the effect of reducing TRAF1 and its signalling partner TRAF2 in lymphoma had on cell proliferation and survival. As shown in FIG. 7, knockdown of TRAF1 or PKN1 in lymphomas had no impact on the tumour growth rate in complete culture medium. This may explain why previous studies have failed to correlate TRAF1 levels with prognosis of lymphoma. When Erwinase was added to the cultures, control treated RAJI showed an arrest in cell growth. Strikingly, PKN1 or TRAF1 knockdown RAJI cells showed substantially more impairment in proliferation after Erwinase treatment, compared to control cells (FIG. 7). To determine whether this effect was due to death of the lymphoma cells, Annexin V and propidium iodide (PI) staining of the lymphomas were analyzed at different times after transfer to Erwinase containing medium. Annexin V binds to phosphatidyl serine, which becomes exposed on the surface of cells as they undergo apoptosis. Then, cells become permeable to propidium iodide upon death. Cells with decreased levels of TRAF1 due to knockdown of TRAF1 or PKN1 demonstrated increased sensitivity to cell death induced by Erwinase, as measured by annexin V and Propidium iodide staining (FIGS. 8 and 9). These findings demonstrate that knockdown of PKN1, which lowers TRAF1 levels, has a similar effect as lowering TRAF1 directly. The slightly greater effect on cell death of direct TRAF1 knockdown compared to PKN1 knockdown is consistent with the smaller effect of PKN1 knockdown on TRAF1 levels compared to direct TRAF1 knockdown with shTRAF1.

Evidence that TRAF1 Serine 146 (S146) is Important in Preventing its Degradation.

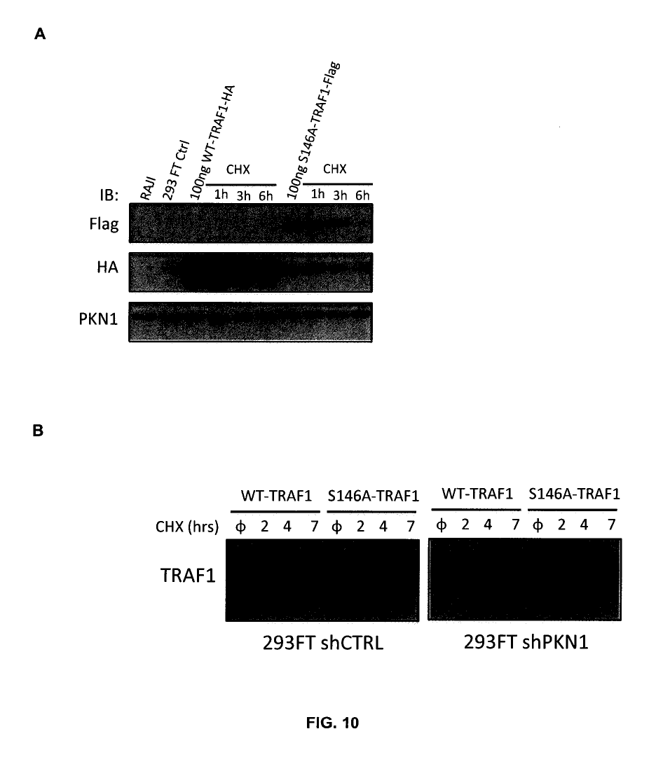

Published work shows that PKN1 can phosphorylate TRAF1 on serine 146.²⁹ To test the role of serine 146 in TRAF1 stabilization by PKN1, serine 146 of TRAF1 was mutated to alanine and expression of HA-tagged WT TRAF1 and Flag-tagged TRAF1 S146A was compared in 293 cells, which constitutively express PKN1. The results show that when the cDNA for WT and S146A TRAF1 are transfected in a 1:1 ratio into 293 cells, TRAF1 S146A exhibits lower protein levels (FIG. 10a). Cycloheximide was also included to block de novo protein translation, so that the decay of TRAF1 in the cell could be assessed, in the absence of new protein synthesis. The results show that WT HA-tagged TRAF1 is stable, whereas Flag-tagged TRAF1 S146A disappears rapidly after cycloheximide treatment. These results show that Serine 146, the target of PKN1 phosphorylation, is required for TRAF1 protein stability. 293 cells express PKN1 constitutively (see lower panels of FIG. 10a). To test the role of PKN1 in TRAF1 stability PKN1 was knocked down in the 293 cells and transfected with WT or S146A TRAF1. The results show that the differences in WT and S146A TRAF1 levels in PKN1 sufficient 293 cells are abrogated when PKN1 is knocked down (FIG. 10b).

TRAF1 Knockdown Lowers the Activity of mTORC1, Thereby Potentiating the Effects of Erwinase.

In an effort to understand the mechanism of synergy between lowering TRAF1 levels and increased sensitivity to Erwinase, the activity of mTORC1, a key sensor of the nutrient state of the cells, was examined. Nutrient stress inhibits the activation of mTORC1, a key regulator of proliferation, metabolism and cell survival.³⁵ PhosphoS6K (pS6K), a downstream target of mTORC1, can be used as a marker of mTORC1 activation. As expected, nutrient stress induced by Erwinase reduced the level of pS6K in the lymphoma cells (FIG. 11). However, unexpectedly, TRAF1 knockdown cells had lower basal level of pS6K prior to treatment, perhaps explaining the synergistic effect of TRAF1 knockdown with Erwinase treatment (FIG. 11A—compare right to left western blots). These findings support the result that lowering TRAF1 levels potentiates the sensitivity of lymphoma to nutrient stress. Further, a correlation between the level of TRAF1 in the cell and the activity of mTORC1 as measured by pS6 levels was shown (FIG. 11C). This suggests that by maintaining/regulating the activity of mTORC1, TRAF1 may partially ameliorate growth arrest induced by nutrient limitation.

Evidence for TRAF1 as a Predictive Biomarker of Resistance to Erwinase.

PKN1 stabilizes TRAF1 and TRAF1 serine 146, a target of PKN1 is required for TRAF1 stability (see above). As lowering TRAF1 by PKN1 knockdown increases sensitivity to nutrient stress, knockdown of PKN1 with RNA interference or anti-sense approaches, or inhibition of PKN1 using chemical inhibitors could be used to increase sensitivity to nutrient stress. In order to develop this strategy, biomarker needs to be developed to predict which cancers would be susceptible. To this end, TRAF1 protein expression was measured in a panel of human lymphomas and the CLL cell line MEC2 by flow cytometry and these levels were correlated with sensitivity to the nutrient stress-inducing drug Erwinase. The results show that cells with very low TRAF1 expression do not fit this correlation, likely because they have evolved other means, besides TRAF1 to avoid regulation by nutrient stress. However, in cells that express TRAF1 above an MFI of 1500, there was a strong correlation between TRAF1 levels and resistance to cell death induced by nutrient stress (FIG. 13). Cancers with these properties are candidates for therapy with PKN1 in combination with nutrient stress.

PKN1 as a Target for Drug Discovery.

PKN1 is a kinase and as such, it belongs to a class of targets that are considered “druggable” due to the presence of active site cleft that can be targeted. In addition, specific PKN1 inhibition is likely to have favourable toxicity, as PKN1 knockout mice are viable and have only a mild inflammatory phenotype (increased germinal centers).³⁶ Of interest, TRAF1 and PKN1 have relatively limited tissue distribution, with highest expression in immune cells and immune related cancers. The fact that they also have a similar tissue distribution (see http://biogps.org/#goto=welcome) supports the hypothesis that PKN1 is a TRAF1 regulator.

Assay to Identify PKN1 Inhibitor that Synergizes with Erwinase:

FIG. 7 provides a rationale for a screen for a PKN1 inhibitor that synergizes with Erwinase: In particular, the assay is used to look for inhibitors that act at nanomolar concentration to reduce cell survival. In the assay, a concentration and duration of Erwinase (1 IU/ml, 48 hr) that on its own has minimal impact in the assay is used (FIG. 7). Inhibitors that reduce the signal in the MTT assay by at least 3 fold relative to Erwinase alone will be identified as potential PKN1 inhibitors. This initial screen is amenable to a 96 well plate assay in which each drug candidate is tested on control RAJI cells with and without Erwinase as well as shPKN1 RAJI cells with Erwinase. Candidate drugs that lower the signal on control RAJI cells only in the presence but not in the absence of Erwinase, and have no effect on PKN1 knockdown cells treated with Erwinase, represent candidates for kinase inhibitors that act through PKN1 inhibition to reduce TRAF1 levels. The candidates can then be verified by determining the inhibitory effects on TRAF1 levels.

TRAF2 knockdown also decreases RAJI survival in the presence of Erwinase (FIG. 14). Knockdown of TRAF2 by shRNA (Mature antisense: ATGGCGTGGAATCTGCAAGGG SEQ ID NO:4) caused increased susceptibility to Erwinase-mediated nutrient stress. Moreover, TRAF2 knockdown cells had lower TRAF1 levels, and consistent with TRAF1 or PKN1 knockdown, they exhibited constitutive reduction in NF-κB and ERK activation. The figure shows flow cytometry profile for TRAF1 levels, pERK levels or phopho P65 subunit of NFkB as indicated on the X-axis. On the Y axis, is indicated knockdown of TRAF2, PKN1, TRAF1 or control sample. The vertical dashed line indicates the peak level of expression in the control sample for comparison with the knockdown cells.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Locksley R M, Killeen N, Lenardo M J. The TNF and TNF receptor superfamilies: integrating mammalian biology. Cell 2001, 104(4): 487-501.
2. Aggarwal B B. Signalling pathways of the TNF superfamily: a double-edged sword. Nat Rev Immunol 2003, 3(9): 745-756.
3. Zheng C, Kabaleeswaran V, Wang Y, Cheng G, Wu H. Crystal structures of the TRAF2: cIAP2 and the TRAF1: TRAF2: cIAP2 complexes: affinity, specificity, and regulation. Mol Cell 2010, 38(1): 101-113.
4. Sabbagh L, Srokowski C C, Pulle G, Snell L M, Sedgmen B J, Liu Y, et al. A critical role for TNF receptor-associated factor 1 and Bim down-regulation in CD8 memory T cell survival. Proc Natl Acad Sci USA 2006, 103(49): 18703-18708.
5. Sabbagh L, Pulle G, Liu Y, Tsitsikov E N, Watts T H. ERK-Dependent Bim Modulation Downstream of the 4-1 BB-TRAF1 Signaling Axis Is a Critical Mediator of CD8 T Cell Survival In Vivo. J Immunol 2008, 180(12): 8093-8101.
6. McPherson A J, Snell L M, Mak T W, Watts T H. Opposing roles for TRAF1 in the alternative versus classical NF-kappaB pathway in T cells. The Journal of biological chemistry 2012, 287(27): 23010-23019.
7. Arron J R, Pewzner-Jung Y, Walsh M C, Kobayashi T, Choi Y. Regulation of the subcellular localization of tumor necrosis factor receptor-associated factor (TRAF)2 by TRAF1 reveals mechanisms of TRAF2 signaling. J Exp Med 2002, 196(7): 923-934.
8. Xie P, Hostager B S, Munroe M E, Moore C R, Bishop G A. Cooperation between TNF receptor-associated factors 1 and 2 in CD40 signaling. J Immunol 2006, 176(9): 5388-5400.
9. Graham J P, Arcipowski K M, Bishop G A. Differential B-lymphocyte regulation by CD40 and its viral mimic, latent membrane protein 1. Immunological reviews 2010, 237(1): 226-248.
10. Zapata J M, Krajewska M, Krajewski S, Kitada S, Welsh K, Monks A, et al. TNFR-associated factor family protein expression in normal tissues and lymphoid malignancies. J Immunol 2000, 165(9): 5084-5096.
11. Cerhan J R, Ansell S M, Fredericksen Z S, Kay N E, Liebow M, Call T G, et al. Genetic variation in 1253 immune and inflammation genes and risk of non-Hodgkin lymphoma. Blood 2007, 110(13): 4455-4463.
12. Baxendale A J, Dawson C W, Stewart S E, Mudaliar V, Reynolds G, Gordon J, et al. Constitutive activation of the CD40 pathway promotes cell transformation and neoplastic growth. Oncogene 2005, 24(53): 7913-7923.
13. Challa A, Eliopoulos A G, Holder M J, Burguete A S, Pound J D, Chamba A, et al. Population depletion activates autonomous CD154-dependent survival in biopsylike Burkitt lymphoma cells. Blood 2002, 99(9): 3411-3418.
14. Furman R R, Asgary Z, Mascarenhas J O, Liou H C, Schattner E J. Modulation of NF-kappa B activity and apoptosis in chronic lymphocytic leukemia B cells. J Immunol 2000, 164(4): 2200-2206.
15. Pham L V, Tamayo A T, Yoshimura L C, Lo P, Terry N, Reid P S, et al. A CD40 Signalosome anchored in lipid rafts leads to constitutive activation of NF-kappaB and autonomous cell growth in B cell lymphomas. Immunity 2002, 16(1): 37-50.
16. Eliopoulos A G, Waites E R, Blake S M, Davies C, Murray P, Young L S. TRAF1 is a critical regulator of JNK signaling by the TRAF-binding domain of the Epstein-Barr virus-encoded latent infection membrane protein 1 but not CD40. J Virol 2003, 77(2): 1316-1328.
17. Horie R, Watanabe T, Morishita Y, Ito K, Ishida T, Kanegae Y, et al. Ligand-independent signaling by overexpressed CD30 drives NF-kappaB activation in Hodgkin-Reed-Sternberg cells. Oncogene 2002, 21(16): 2493-2503.
18. Zhang B, Wang Z, Li T, Tsitsikov E N, Ding H F. NF-kappaB2 mutation targets TRAF1 to induce lymphomagenesis. Blood 2007, 110(2): 743-751.
19. Jones R G, Thompson C B. Revving the engine: signal transduction fuels T cell activation. Immunity 2007, 27(2): 173-178.
20. Vander Heiden M G, Cantley L C, Thompson C B. Understanding the Warburg effect: the metabolic requirements of cell proliferation. Science 2009, 324(5930): 1029-1033.
21. DeBerardinis R J, Mancuso A, Daikhin E, Nissim I, Yudkoff M, Wehrli S, et al. Beyond aerobic glycolysis: transformed cells can engage in glutamine metabolism that exceeds the requirement for protein and nucleotide synthesis. Proceedings of the National Academy of Sciences of the United States of America 2007, 104(49): 19345-19350.
22. Wise D R, Thompson C B. Glutamine addiction: a new therapeutic target in cancer. Trends Biochem Sci 2010, 35(8): 427-433.
23. Carr E L, Kelman A, Wu G S, Gopaul R, Senkevitch E, Aghvanyan A, et al. Glutamine uptake and metabolism are coordinately regulated by ERK/MAPK during T lymphocyte activation. J Immunol 2010, 185(2): 1037-1044.
24. Daye D, Wellen K E. Metabolic reprogramming in cancer: Unraveling the role of glutamine in tumorigenesis. Seminars in cell & developmental biology 2012.
25. Pieters R, Hunger S P, Boos J, Rizzari C, Silverman L, Baruchel A, et al. L-asparaginase treatment in acute lymphoblastic leukemia: a focus on Erwinia asparaginase. Cancer 2011, 117(2): 238-249.
26. Reinert R B, Oberle L M, Wek S A, Bunpo P, Wang X P, Mileva I, et al. Role of glutamine depletion in directing tissue-specific nutrient stress responses to L-asparaginase. The Journal of biological chemistry 2006, 281(42): 31222-31233.
27. Li X, Yang Y, Ashwell J D. TNF-RII and c-IAP1 mediate ubiquitination and degradation of TRAF2. Nature 2002, 416(6878): 345-347.
28. Duckett C S, Thompson C B. CD30-dependent degradation of TRAF2: implications for negative regulation of TRAF signaling and the control of cell survival. Genes Dev 1997, 11(21): 2810-2821.
29. Kato T, Jr., Gotoh Y, Hoffmann A, Ono Y. Negative regulation of constitutive NF-kappaB and JNK signaling by PKN1-mediated phosphorylation of TRAF1. Genes Cells 2008, 13(5): 509-520.
30. Guo F, Sun A, Wang W, He J, Hou J, Zhou P, et al. TRAF1 is involved in the classical NF-kappaB activation and CD30-induced alternative activity in Hodgkin's lymphoma cells. Mol Immunol 2009.
31. Sabbagh L, Andreeva D, Laramee G D, Oussa N A, Lew D, Bisson N, et al. Leukocyte-specific protein 1 links TNF receptor-associated factor 1 to survival signaling downstream of 4-1BB in T cells. J Leuk Biol 2013, 93(5): 713-721.
32. Ma L, Chen Z, Erdjument-Bromage H, Tempst P, Pandolfi P P. Phosphorylation and functional inactivation of TSC2 by Erk implications for tuberous sclerosis and cancer pathogenesis. Cell 2005, 121(2): 179-193.
33. Levis M, Ravandi F, Wang E S, Baer M R, Perl A, Coutre S, et al. Results from a randomized trial of salvage chemotherapy followed by lestaurtinib for patients with FLT3 mutant AML in first relapse. Blood 2011, 117(12): 3294-3301.
34. Hexner E O, Serdikoff C, Jan M, Swider C R, Robinson C, Yang S, et al. Lestaurtinib (CEP701) is a JAK2 inhibitor that suppresses JAK2/STAT5 signaling and the proliferation of primary erythroid cells from patients with myeloproliferative disorders. Blood 2008, 111(12): 5663-5671.
35. Laplante M, Sabatini D M. mTOR signaling in growth control and disease. Cell 2012, 149(2): 274-293.
36. Yasui T, Sakakibara-Yada K, Nishimura T, Morita K, Tada S, Mosialos G, et al. Protein kinase N1, a cell inhibitor of Akt kinase, has a central role in quality control of germinal center formation. Proceedings of the National Academy of Sciences of the United States of America 2012, 109(51): 21022-21027.

1. A method of treating a lymphocyte related cancer in a subject comprising administering a nutrient stress inducing agent in combination with an agent that lowers the levels of or inhibits TRAF1 to the subject in need thereof.

2. The method of claim 1, wherein the lymphocyte related cancer is lymphoma or leukemia.

3. (canceled)

4. The method of claim 1, wherein the nutrient stress inducing agent is asparaginase or arginase.

5. The method of claim 4, wherein the asparaginase is Erwinase.

6. (canceled)

7. The method of claim 1, wherein the agent that lowers the levels of or inhibits TRAF1 is an antisense or shRNA to TRAF1 or is a TRAF1 antibody.

8. The method of claim 7, wherein the shRNA comprises the nucleotide sequence as shown in SEQ ID NO:1 or a variant thereof.

9. The method of claim 1, wherein the agent that lowers the levels of or inhibits TRAF1 is a PKN1 inhibitor.

10. The method of claim 9, wherein the PKN1 inhibitor is an antisense or shRNA to PKN1 or is a PKN1 antibody.

11. The method of claim 10, wherein the shRNA comprises the nucleotide sequence as shown in SEQ ID NO:2 or a variant thereof.

12. (canceled)

13. A method of determining treatment for a lymphocyte-related cancer in a subject comprising:
determining whether the lymphocyte-related cancer is TRAF1 low or TRAF1 high by comparing to a control;
wherein if the lymphocyte related cancer is TRAF1 low it is indicative of treating the subject with a nutrient stress inducing agent; and wherein if the lymphocyte related cancer is TRAF1 high, it is indicative of treating the subject with a nutrient stress inducing agent in combination with an agent that lowers the levels of or inhibits TRAF1.

14. A method of determining treatment for a lymphocyte-related cancer in a subject comprising:
a) determining whether the lymphocyte-related cancer is TRAF1 low or TRAF1 high by comparing to a control;
b) treating the subject with a nutrient stress inducing agent if the subject is TRAF1 low; and
c) treating the subject with a nutrient stress inducing agent in combination with an agent that lowers the levels of or inhibits TRAF1 if the subject is TRAF1 high.

15. The method of claim 13, wherein the control is a TRAF1 high control from the same type of tumour or from a pool of positive tumours of the same type or wherein the control is a TRAF1 low control from a cell line of a similar type of tumour that has been treated with shPKN1 or shTRAF1.

16. (canceled)

17. (canceled)

18. The method of claim 13 wherein determining whether the lymphocyte-related cancer is TRAF1 low or TRAF1 high comprises measuring the expression of TRAF1 from a sample.

19. The method of claim 18, wherein determining whether the cancer is TRAF1 low or TRAF1 high further comprises assaying the expression level of Raji control cells or Daudi control cells at the same time as measuring the level of TRAF1 in the sample.

20. The method of claim 18, wherein the sample is peripheral blood mononuclear cells, whole blood samples or from a biopsy.

21. (canceled)

22. The method of claim 13, wherein the nutrient stress-inducing agent is an asparaginase, arginase or glutaminase.

23. The method of claim 22, wherein the nutrient stress-inducing agent is Erwinase.

24. The method of claim 13, wherein the lymphocyte related cancer is lymphoma or leukemia.

25. (canceled)

26. (canceled)

27. (canceled)
